(19) United States
(12) Patent Application Publication
(10) Pub. No.: US 2005/0287085 A1
(43) Pub. Date: Dec. 29, 2005

(54) Composition for improving the physico-chemical properties of apatite-based materials, uses thereof and method using same

(75) Inventors: Jean Louis Cazor, Paris (FR); Martin Crootveld, Kent (GB); Francois Lhuisset, Gentilly (FR); Pascale Robineau, Palaiseau (FR); Christopher Silwood, Enfield Middlesex (GB); Maurice Sofeir, Paris (FR)

Correspondence Address: DENTSPLY INTERNATIONAL INC, 570 WEST COLLEGE AVENUE, YORK, PA, 17404, US

(73) Assignee: DENTSPLY INTERNATIONAL INC., York (PA)

(21) Appl. No.: 10/526,581

(22) PCT Filed: Sep. 4, 2003
(86) PCT No.: PCT/FR03/02643

§ 371 Date: Jun. 15, 2005

(30) Foreign Application Priority Data
Sep. 6, 2002 (FR) 02/11062
Mar. 21, 2003 (FR) 03/03472

(51) Int. Cl. A61K 7/18; A61K 31/28

(52) U.S. Cl. 424/52; 514/492

(57) ABSTRACT

The invention concerns a composition comprising at least one titanium and fluorine derivative for reinforcing materials based on natural or synthetic apatite, in particular teeth, bones, dental implants and surgical prostheses. The inventive compositions enable in particular apatite materials to be enriched with titanium and fluorine. The invention also concerns a method for reinforcing apatite-based materials using said compositions, and use of titanium and fluorine derivatives to fight against staining of apatite-based material.

The invention relates to a composition intended to improve the physicochemical characteristics of natural or artificial apatite-based materials, in particular of implants. It also relates to the use of titanium derivatives for the strengthening of apatite-based materials and to a process for the strengthening of apatite-based materials.

It also relates to the use of derivatives of titanium and of fluorine for protecting apatite-based materials from coloration, in particular extrinsic colorations, and to a process for protecting apatite-based materials from coloration.

A high proportion of prostheses or implants for dental or medical use is manufactured from titanium.

The advantage of titanium in the preparation of such prostheses or implants is well known. Its mechanical properties, its high resistance to corrosion and its lightness make it the material of choice for these applications.

In addition, titanium shows very good compatibility with biological tissues. It generally does not detrimentally affect the growth of osteoblasts, fibroblasts and gingival epithelial cells and is commonly used in the manufacture of dental or bone prosthesis for replacing failing tissues, including intervertebral discs and the ossicles of the ear.

The very good compatibility between titanium and apatite-based tissues is explained in part by the formation, at the surface of the titanium, of a fine layer of titanium oxide capable of protecting the underlying metal and of being bonded to the calcium atoms and to the phosphorous groups of the apatite.

Furthermore, the visible part of the tooth has a natural colour which changes over time. This colour depends in particular on the whiteness and on the transparency of the dental enamel and on the colour of the underlying dentine.

The whiteness of the enamel and the dentine is generally in inverse proportion to the degree of mineralization of their main constituent, hydroxyapatite. Thus, in the absence of abnormality, the tooth then has an ivory-white colour. However, various phenomena occur over time and have an influence on the colour of the teeth.

Thus, it is known that mineralization of the enamel and of the dentine increases throughout the life of the tooth, rendering it less white and less transparent.

The enamel is also subjected to erosion by acid substances and by dental caries; to mechanical abrasion and wear. These factors contribute to reducing its brightness.

Finally, extrinsic colorations can modify the colour of the tooth. For example, some foodstuffs, such as coffee, tea or blueberries, comprise coloured pigments which can become attached to the surface of the teeth and can detrimentally affect their hue. Other substances, such as the tars present in cigarettes, or some medical treatments can act in the same way and can modify the colour of the teeth.

The document WO 0105797 discloses titanium derivatives and their use in compositions for buccal use, as agent for protecting against dental caries. This document discloses that the titanium derivative forms a protective layer at the surface of the tooth, in the form of a glaze, under pH conditions varying from approximately 6.5 to approximately 7.5.

An aim of the invention is to provide a novel composition for strengthening apatite-based materials, comprising compounds derived from titanium and from fluorine, which is capable of modifying the structure of the apatite.

A first object of the invention thus relates to such a composition.

Another object of the invention relates to the use of derivatives of titanium and of fluorine for strengthening apatite-based materials.

Another object of the invention relates to a process for strengthening apatite-based materials.

Another object of the invention relates to a novel use of compounds derived from titanium and from fluorine for protecting apatite-based materials from colorations, in particular extrinsic colorations.

Another object of the invention relates to a process for protecting apatite-based materials from colorations.

In the context of the present invention, the term “apatite-based materials” is understood to mean natural hydroxyapatites, in particular dental enamel, dentine or bones, and artificial ceramics based on calcium phosphate intended for medical applications, in particular dental implants, percutaneous or periodontal implant devices, or bone prostheses used in particular in orthopaedic, maxillofacial or spinal surgery.

The composition intended to strengthen apatite-based materials according to the invention is characterized in that it comprises at least one derivative of titanium and of fluorine corresponding to the general formula (I) below:
TiFxRy (I)
in which
x is an integer varying from 1 to 6 and y is 0, 1 or 2, with the condition that, when y is 0, x does not represent 4,
and R represents:
   a component chosen from K, Na or NH₄, or
   a ligand L of formula (11) below: COOH 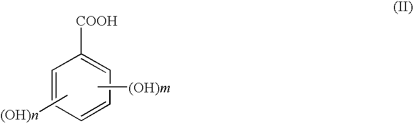
      in which m is 0 or 1 and n is 0, 1 or 2;
      the composition according to the invention being characterized in addition in that it has, in the dissolved state, preferably dissolved in an aqueous medium, a pH of less than or equal to 6.

Ligands L which can be used are in particular benzoic acid derivatives, in particular 2-hydroxybenzoic acid of formula (IV) below and its derivatives:

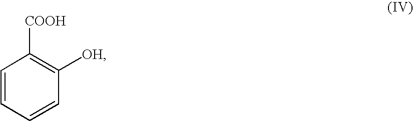

3-hydroxybenzoic acid of formula (V) below and its derivatives:

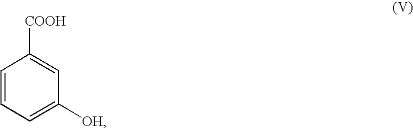

4-hydroxybenzoic acid of formula (VI) below and its derivatives:

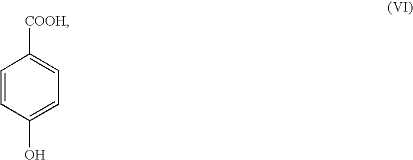

2,3-dihydroxybenzoic acid of formula (VII) below and its derivatives:

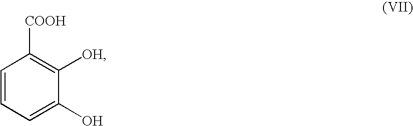

3,4-dihydroxybenzoic acid of formula (VIII) below and its derivatives:

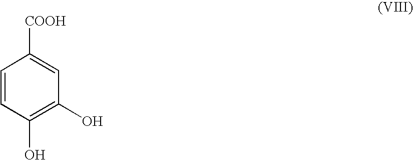

The compounds in which R represents a ligand of formula (11) can comprise one or more asymmetric carbon atoms. They can therefore exist in the form of enantiomers or of diastereoisomers. These enantiomers, diastereoisomers and their mixtures, including racemic mixtures, form part of the invention.

The compounds in which R represents a ligand of formula (11) can exist in the form of bases or of addition salts with acids. Such addition salts form part of the invention.

These salts are advantageously prepared with pharmaceutically acceptable acids but the salts of other useful acids, for example useful in the purification or isolation of the compounds, also form part of the invention.

The compounds in which R represents a ligand of formula (II) can also exist in the form of hydrates or of solvates, namely in the form of associations or of combinations with one or more molecules of water or with a solvent. Such hydrates and solvates also form part of the invention.

A first example of a compound derived from titanium and from fluorine according to the invention, obtained by using 2-hydroxybenzoic acid as ligand L, is the compound represented by the formula (III):

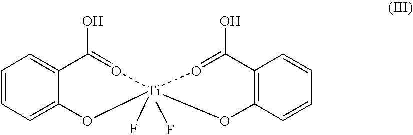

Another example of a derivative of titanium and of fluorine according to the invention in which R represents an inorganic compound is potassium hexafluorotitanate of formula K₂TiF₆.

Other examples of compounds which can be used in the context of the invention are sodium hexafluorotitanate of formula Na₂TiF₆ and ammonium hexafluorotitanate of formula (NH₄)₂TiF₆.

The compounds which can be used in the context of the invention, when their method of preparation is not described, are commercially available or described in the literature, or else can be prepared according to methods which are described therein or which are known to a person skilled in the art.

The derivatives of titanium and of fluorine in which R represents a ligand L of formula (II) above can be prepared according to the method disclosed in the document WO 0105797.

The compositions of the invention have a titanium content varying from approximately 10 to approximately 1000 ppm, preferably approximately 300 ppm, and a content of fluorine ions varying from approximately 50 to approximately 1500 ppm, preferably approximately 240 ppm.

According to one embodiment, the compositions according to the invention furthermore comprise an additional fluorinated compound, in particular a fluorine salt, for example sodium fluoride or sodium monofluorophosphate.

This additional fluorinated compound is present in the compositions of the invention in an amount varying from approximately 50 to approximately 1500 ppm, preferably from approximately 100 to approximately 500 ppm.

The compositions of the invention have, in a dissolved state, a pH of less than or equal to 6, preferably of between approximately 5 and approximately 1, depending on the method of administration of the composition. The dissolution of the compositions of the invention is preferably carried out in an aqueous medium but can also be carried out, depending in particular on the derivative of titanium and of fluorine used, in an organic solvent, such as ethanol.

It has been demonstrated that, at such pH values, the compositions of the invention make possible the substitution of calcium atoms present in the apatite by titanium and the substitution of hydroxyl groups, of bicarbonates or of impurities present in the apatite by fluorine.

This substitution modifies the structure of the apatite and renders it more resistant, not only to acid erosion and to dental caries but also to abrasion, to wear and to traumatic impacts.

The incorporation of an additional fluorinated compound further increases the resistance of the apatite to dissolution by acids.

It is obvious that the invention can be applied not only to dental enamel and to dentine in compositions for buccal use but also to other natural apatites, for example bone tissue, or artificial apatites, for example ceramics.

The compositions of the invention can be provided in various forms conventional for administration in a clinical situation (topical route) or for the preparation of artificial apatites.

In administration by the topical route, the compositions of the invention can be provided in the form, in the case of a buccal application, of a dentifrice, of a powder to be diluted, of a spray, of a chewing gum, of a pastille to be sucked, of a dental gel, of a buccal implant, such as a patch, of a mouthwash or of a solution.

For application to bone or to an artificial apatite, the compositions of the invention can be provided in the form of a solution, of a gel, of a paste or of a powder to be diluted.

All these forms in themselves are well known to a person skilled in the art. In addition to the compounds derived from titanium, which may or may not be combined with a fluorine salt, the forms mentioned above can comprise excipients or ingredients conventional for each of these forms.

For example, the forms for buccal application can comprise anionic, amphoteric, zwitterionic, cationic or nonionic surfactants. They can also comprise thickening agents, cohesion agents, sweetening agents, humectants, refreshing agents, preservatives, colorants, whitening agents, flavouring agents, flavour enhancers, plant essential oils, plasticizing agents, peptizing agents, antitartar agents, agents for inhibiting the production of volatile sulphur compounds, such as zinc salts and complexes, healing substances, agents for combating bleeding, polishing agents, agents for combating dental plaque, such as chlorhexidine, hexetidine, cetylpyridinium chloride or triclosan and/or enzymes, such as dextranase, mutanase, lysozymes, lactoferrin or peroxidases.

Generally, the composition according to the invention, administered by the topical route, comprises compounds derived from titanium in an amount such that its titanium content is greater than 0.001% by weight, preferably between 0.01 and 0.1% by weight, more preferably between 0.01 and 0.05% by weight, with respect to the total weight of the said preparation.

A first example of a composition according to the invention, in the form of a powder to be diluted at the time of use for administration by the topical route, comprises the following components, expressed as percentages by weight with respect to the total weight of the composition:

Composition A:

| | |
|---|---|
| compound of formula III | 9% |
| mannitol | 79% |
| flavouring | 8% |
| saccharin sodium | 4% |

A sachet of 500 mg of the above composition, dissolved in 20 ml of purified water, produces a solution comprising 300 ppm of titanium, with a pH of between 3.5 and 5.

Another example of a composition according to the invention, in the form of a powder to be diluted at the time of use for the preparation of a solution for strengthening artificial apatites, comprises the following components, expressed as percentages by weight with respect to the total weight of the preparation:

Composition B:

| | |
|---|---|
| compound of formula III | 100% |

A sachet of 750 mg of the above composition, dissolved in 100 ml of purified water, produces a solution with a pH of approximately 3 comprising 1000 ppm of titanium.

When the composition of the invention is administered by the topical route, the pH of the composition in the dissolved state is less than or equal to 6, preferably between approximately 5 and approximately 2.

When the composition of the invention is used to strengthen an artificial apatite, the pH of the composition, once dissolved, can be lower than in the case of use by the topical route. Thus, the composition of the invention has, in solution, a pH of less than or equal to 6, preferably of between approximately 4 and approximately 1.

The majority of the compounds derived from titanium and from fluorine which can be used in the invention, for example the compound of formula (III), are acids in aqueous solution.

In some cases, however, it is necessary to adjust the pH of the composition in order for it to be less than or equal to 6. In other cases, it may also be necessary to increase the pH, when the composition exhibits, in the dissolved state, an excessively strong acid nature.

The pH of the composition can be adjusted, according to the relative acidity of the derivatives of titanium and of fluorine used, by additional acidic or alkaline agents, according to circumstances. Such agents are known per se for this use in compositions intended to be administered to man.

For example, acidic agents which can be used are in particular citric acid, hydrochloric acid, lactic acid, phosphoric acid or tartaric acid, and alkaline agents which can be used are in particular sodium hydroxide, monoethanolamine, diethanolamine or triethanolamine.

Another subject-matter of the invention is the use of a composition comprising at least one derivative of titanium and of fluorine for strengthening apatite-based materials.

According to the invention, the composition comprising at least one derivative of titanium and of fluorine is as defined in the preceding part.

The invention is particularly useful for the strengthening of natural or artificial apatite or hydroxyapatite as defined in the preceding part.

Another subject-matter of the invention is a process for strengthening apatite-based materials.

The process of the invention is characterized in that it comprises the stage consisting in applying, to the apatite-based material, a composition comprising a derivative of titanium and of fluorine as defined in the preceding part, the said composition having, in the dissolved state, a pH of less than or equal to 6.

According to the process of the invention, prior to the application of the composition, a stage of treatment with an acidic or demineralizing compound can be carried out. Preferably, this treatment stage is carried out by using an acidic or demineralizing agent, such as citric acid, lactic acid, phosphoric acid or tartaric acid.

According to an alternative embodiment of the process of the invention, the composition applied to the apatite-based material to be strengthened comprises a derivative of titanium and of fluorine as defined in the preceding part and furthermore comprises an additional fluorinated compound, in particular in the salt form, as defined in the preceding part.

Another subject-matter of the invention is the use of compounds derived from titanium and from fluorine for protecting apatite-based materials from colorations, in particular extrinsic colorations.

The invention thus consists of the use of at least one derivative of titanium and of fluorine corresponding to the general formula (1) below:
TiFₓR_{y} (I)
in which
x is an integer varying from 1 to 6 and y is 0, 1 or 2, and R represents:
   a compound chosen from K, Na or NH₄, or
   a ligand L of formula (11) below: 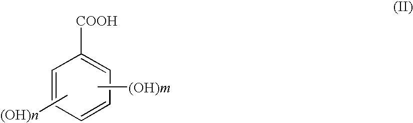
      in which m is 0 or 1 and n is 0, 1 or 2, in the form of the base or of an addition salt with an acid, and in the form of a hydrate or solvate, as agent for combating coloration of apatite-based materials in a composition for buccal use.

The composition for buccal use defined above has, in the dissolved state, preferably dissolved in an aqueous medium, a pH of less than or equal to 6.

The ligands L which can be used are in particular those described in the preceding part, in particular the compounds of formulae (IV), (V), (VI), (VII), (VIII) and their derivatives.

A first example of a compound derived from titanium and from fluorine which can be used in the invention is the compound of formula (III) defined in the preceding part.

Another example of a derivative of titanium and of fluorine which can be used according to the invention in which R represents an inorganic component, in addition to those already described in the preceding part, is titanium tetrafluoride of formula TiF₄.

The compositions for buccal use which can be used for protecting apatite-based materials from colorations are as defined in the preceding part, in particular with regard to their titanium content, their fluorine, the excipients which can be used, their pH and their method of administration.

An example of a composition for buccal use which can be used in the invention, in the form of a powder to be diluted at the time of use for the preparation of a solution for combating coloration of apatites, is composition B as defined in the preceding part.

Another subject-matter of the invention is a process for protecting apatite-based materials from coloration.

The process of the invention is characterized in that it comprises the stage consisting in applying, to the apatite-based material, a composition comprising a derivative of titanium and of fluorine as defined in the preceding part, the said composition having, in the dissolved state, a pH of less than or equal to 6.

According to the process of the invention, prior to the application of the composition, a stage of treatment with an acidic or demineralizing compound can be carried out.

Preferably, this treatment stage is carried out by using an acidic or demineralizing agent, such as citric acid, lactic acid, phosphoric acid or tartaric acid.

According to an alternative embodiment of the process of the invention, the composition applied to the apatite-based material comprises a derivative of titanium and of fluorine as defined in the preceding part and furthermore comprises an additional fluorinated compound, in particular in the salt form, as defined in the preceding part.

The aim of the examples which follow is to illustrate the invention.

### EXAMPLE 1

Compositions 1 and 2 according to the invention are prepared in order to study the effects of the compositions of the invention on the strengthening of hydroxyapatite:

Composition 1: Aqueous potassium hexafluorotitanate solution (comprising 200, 400 or 1000 ppm of titanium) for local application.

Composition 2: Aqueous solution of compound of formula III (comprising 200, 300, 400 or 1000 ppm of titanium) for local application.

Modification of the Structure of Hydroxyapatite

The above compositions (with a pH of between 1 and 6) are brought into contact with hydroxyapatite powder purchased commercially.

The composition of the hydroxyapatite after treatment is studied by X-ray photoelectron spectroscopy.

The results obtained are shown in Table I below.

TABLE I

| | Ca | O | Ti | F | Ti/Ca | F/O |
|---|---|---|---|---|---|---|
| C | 21.4 | 63.9 | | | | |
| X1 | 18.2 | 58.5 | 3.5 | 6.3 | 0.19 | 0.11 |
| X2 | 18.3 | 57.1 | 3.8 | 7.5 | 0.21 | 0.13 |
| Y1 | 14.2 | 61.0 | 6.2 | 5.5 | 0.44 | 0.09 |
| Y2 | 13.0 | 60.3 | 7.3 | 6.0 | 0.56 | 0.10 |

C: control hydroxyapatite (treated with water)
X1 et X2: hydroxyapatite treated with composition 1 (solutions comprising 200 and 400 ppm of titanium respectively)
Y1 et Y2: hydroxyapatite treated with composition 2 (solutions comprising 200 and 400 ppm of titanium respectively)

These results show that the structure of the hydroxyapatite is depleted in calcium and in oxygen in contact with the compositions of the invention, and is enriched in titanium and in fluorine, the more so as the solution applied increases in concentration.

The analysis of the samples of treated hydroxyapatite is continued by mass spectrometry. The percentage of calcium substituted by titanium and the percentage of hydroxyl groups substituted by fluorine are compared with those obtained after bringing the hydroxyapatite into contact with the sodium fluoride solution.

The results obtained are shown in Table II below.

TABLE II

| Treatment of the | | |
|---|---|---|
| hydroxyapatite | Substitution of Ca by Ti | Substitution of OH by F |
| NaF | 0.3% | 52.0% |
| X3 | 17.1% | 63.0% |
| Y3 | 40.3% | 55.3% |

X3: Composition 1 comprising 1000 ppm of titanium
Y3: Composition 2 comprising 1000 ppm of titanium
NaF: Sodium fluoride with a fluorine content of 1000 ppm.

These results show the simultaneous substitution of calcium by titanium and of hydroxyl groups by fluorine with the solutions tested.

Modification of the Composition of the Dental Enamel after Topical Application

To evaluate the effects of the treatment by local application, freshly extracted human teeth are brought into contact with Composition 2. On each tooth, an experimental window is defined and separated into two parts. A moderate acidic attack (dilute phosphoric acid for 1 minute) is carried out on the window. One of the halves of the window is subsequently isolated under wax, while the other half is exposed to the treatment with Composition 3 comprising 300 ppm of titanium.

The enamel situated under the treated half is examined by scanning electron microscopy. Its titanium composition is determined by elemental analysis using an X-ray photoelectron spectroscope. The results demonstrate a modification in the hydroxyapatite constituting the dental enamel, related to the in depth incorporation of titanium in the structure.

The results show that the invention makes possible not only the substitution of the hydroxyl groups by fluorine but also the substitution of calcium by titanium under conditions of local application of the composition of the invention, in an acid medium.

### EXAMPLE 2

To study the effects of compounds derived from titanium and from fluorine on the extrinsic colorations of the tooth, the following composition was prepared:
0.2% aqueous solution of the compound of formula III (corresponding to 0.036% of titanium)

The solution is used in local application, at a pH of approximately 4.6.

In Vitro Effects on the Precursors of the Coloration

Use is made, as coloration model, of a precursor based on lysine in the presence of a reducing sugar, which produces brown-coloured melanoids by reaction.

An aqueous solution comprising 100 mM of lysine and 100 mM of reducing sugar is prepared. The aqueous solution of the compound of formula III prepared above is mixed (50/50 v/v mixture) with the solution of lysine and of reducing sugar, equilibrated at 60° C. for 3 days and then diluted (1/1 v/v), either in water (solution 1), or in artificial saliva (solution 2). Control mixtures, in which the solution of the compound of formula III is replaced by water, are also prepared in the same way (solutions 3 and 4, respectively). The appearance of the melanoid chromophore in the mixture is measured by spectrophotometry for 15 days.

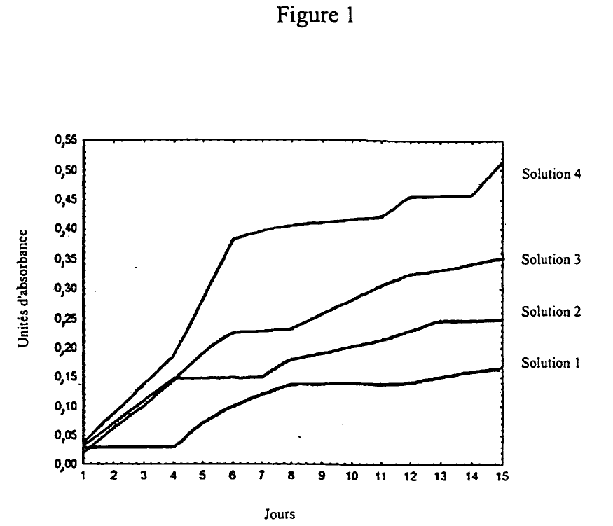

The results obtained are shown in FIG. 1.

The absorbance is measured at 500 nm. It is apparent that, in vitro, the solution comprising the derivative of titanium and of fluorine inhibits the formation of coloured melanoid chromophore from lysine and reducing sugar.

Analogous results are obtained when iron or tin is used in the presence of a source of sulphide ions as coloration precursors.

### EXAMPLE 3

Protection of the Tooth ex vivo from Coloration

To evaluate the effects of the treatment by local application, freshly extracted human teeth are brought into contact with the same solution as in Example 1. An experimental window is defined on each tooth and the remaining region is masked under wax. The teeth are subsequently exposed to the treatment with the same solution as in Example 1.

After rinsing, the wax is removed and the teeth are immersed in aqueous solution comprising lysine and reducing sugar, according to the model described in Example 1, at 60° C. for 2 days.

The results show that the parts of the teeth treated with the solution comprising the derivative of titanium and of fluorine have not undergone significant coloration, whereas the untreated regions are strongly coloured brown.

The protection conferred by the derivative of titanium and of fluorine under the conditions described is thus demonstrated directly on the tooth.

1. Composition intended to strengthen apatite-based materials, characterized in that it comprises at least one derivative of titanium and of fluorine corresponding to the general formula (I) below:
TiFxRy (I)
in which
x is an integer varying from 1 to 6 and y is 0, 1 or 2, with the condition that, when y is 0, x does not represent 4,
and r represents:
a component chosen from K, Na or NH₄, or
a ligand L of formula (II) below: 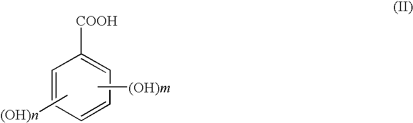
in which m is 0 or 1 and n is 0, 1 or 2; in the form of the base or of an addition salt with an acid, and in the form of a hydrate or solvate,
the composition being additionally characterized in that it has, in the dissolved state, preferably dissolved in an aqueous medium, a pH of less than or equal to 6.

2. Composition according to claim 1, characterized in that the ligands L are in particular benzoic acid derivatives, in particular 2-hydroxybenzoic acid of formula (IV) below and its derivatives: 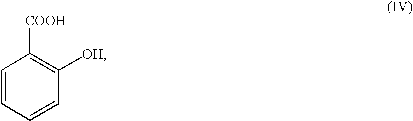
3-hydroxybenzoic acid of formula (V) below and its derivatives: 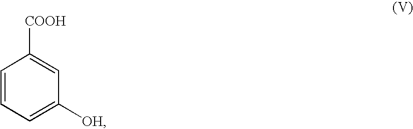
4-hydroxybenzoic acid of formula (VI) below and its derivatives: 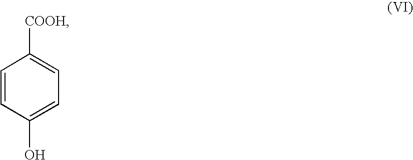
2,3-dihydroxybenzoic acid of formula (VII) below and its derivatives: 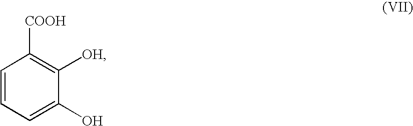
3,4-dihydroxybenzoic acid of formula (VIII) below and its derivatives:

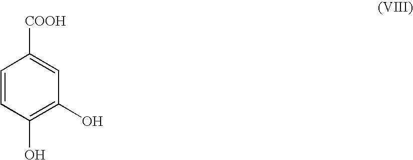

3. Composition according to claim 1, characterized in that the compound derived from titanium and from fluorine is the compound represented by the following formula (III):

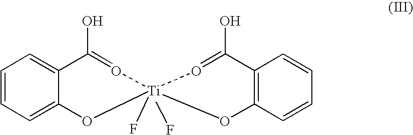

4. Composition according to claim 1, characterized in that the derivative of titanium and of fluorine is in particular potassium hexafluorotitanate of formula K₂TiF₆, sodium hexafluorotitanate of formula Na₂TiF₆ or ammonium hexafluorotitanate of formula (NH₄)₂TiF₆.

5. Composition according to any one of claims 1 to 4, characterized in that it has a titanium content varying from approximately 10 to approximately 1000 ppm, preferably approximately 300 ppm, and a content of fluorine ions varying from approximately 50 to approximately 1500 ppm, preferably approximately 240 ppm.

6. Composition according to any one of claims 1 to 5, characterized in that it comprises, for topical administration, a compound derived from titanium and from fluorine in an amount such that the titanium content is greater than 0.001% by weight, preferably between 0.01 and 0.1% by weight, more preferably between 0.01% and 0.05% by weight, with respect to the total weight of the said composition.

7. Composition according to any one of claims 1 to 6, characterized in that it furthermore comprises an additional fluorinated compound, in particular a fluorine salt, for example sodium fluoride or sodium monofluorophosphate, in an amount varying from approximately 50 ppm to approximately 1500 ppm, preferably from approximately 100 ppm to approximately 500 ppm.

8. Composition according to any one of claims 1 to 7, characterized in that it is administered by the topical route in the form of a dentifrice, of a powder to be diluted, of a spray, of a chewing gum, of a pastille to be sucked, of a gel, of a buccal implant, such as a patch, of a mouthwash or of a solution.

9. Use of at least one compound derived from titanium and from fluorine corresponding to the general formula (I) below:
TiFxRy (I)
in which
x is an integer varying from 1 to 6 and y is 0, 1 or 2, with the condition that, when y is 0, x does not represent 4
and R represents:
a compound chosen from K, Na or NH₄, or
a ligand L of formula (II) below: 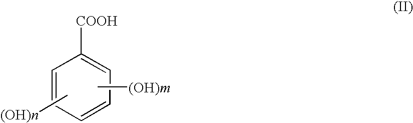
in which m is 0 or 1 and n is 0, 1 or 2, in the form of a base or of an addition salt with an acid, and in the form of a hydrate or solvate, for strengthening apatite-based materials.

10. Use according to claim 9, characterized in that the composition is as defined in any one of claims 2 to 8.

11. Use according to claim 9 or 10 for strengthening natural hydroxyapatites, in particular dental enamel, dentine or bones, and artificial ceramics based on calcium phosphate intended for medical applications, in particular dental implants, percutaneous or periodontal implant devices, or bone prostheses used in orthopaedic, maxillofacial or spinal surgery.

12. Process for strengthening apatite-based materials, comprising the stage consisting in applying, to the apatite-based material, a composition comprising a derivative of titanium and of fluorine as defined in any one of claims 1 to 8, the said composition having, in the dissolved state, a pH of less than or equal to 6.

13. Process according to claim 12, characterized in that, prior to the application of the composition, a stage of treatment with an acidic or demineralizing compound is carried out.

14. Process according to claim 13, characterized in that the acidic or demineralizing compound is in particular citric acid, lactic acid, phosphoric acid or tartaric acid.

15. Use of at least one derivative of titanium and of fluorine corresponding to the general formula (I) below:
TiFxRy (I)
in which
x is an integer varying from 1 to 6 and y is 0, 1 or 2, and R represents:
a compound chosen from K, Na or NH₄, or
a ligand L of formula (II) below: 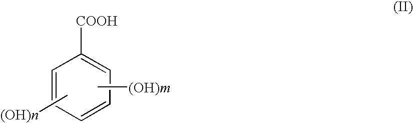
in which m is 0 or 1 and n is 0, 1 or 2; in the form of a base or of an addition salt with an acid, and in the form of a hydrate or solvate, as agent for combating coloration of apatite-based materials in a composition for buccal use.

16. Use according to claim 15, characterized in that the composition has, in the dissolved state, preferably dissolved in an aqueous medium, a pH of less than or equal to 6.

17. Use according to claim 15 or 16, characterized in that the ligands L are in particular benzoic acid derivatives, in particular 2-hydroxybenzoic acid of formula (IV) below and its derivatives: 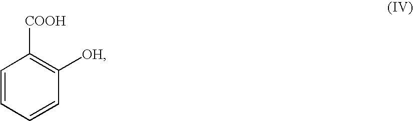
3-hydroxybenzoic acid of formula (V) below and its derivatives: 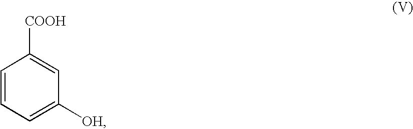
4-hydroxybenzoic acid of formula (VI) below and its derivatives: 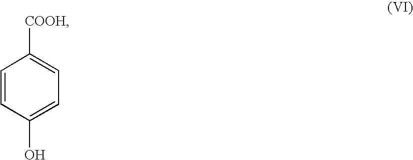
2,3-dihydroxybenzoic acid of formula (VII) below and its derivatives: 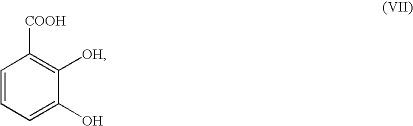
3,4-dihydroxybenzoic acid of formula (VII) below and its derivatives:

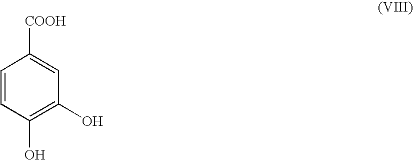

18. Use according to claim 15 or 16, characterized in that the compound derived from titanium and from fluorine is the compound represented by the following formula (III):

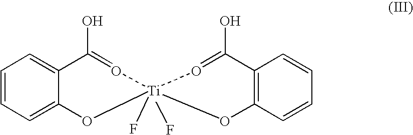

19. Use according to claim 15, characterized in that the derivative of titanium and of fluorine is in particular titanium tetrafluoride of formula TiF₄, potassium hexafluorotitanate of formula K₂TiF₆, sodium hexafluorotitanate of formula Na₂TiF₆ or ammonium hexafluorotitanate of formula (NH₄)₂TiF₆.

20. Use according to any one of claims 15 to 19, characterized in that the composition has a titanium content varying from approximately 10 to approximately 1000 ppm, preferably approximately 300 ppm, and a content of fluorine ions varying from approximately 50 to approximately 1500 ppm, preferably approximately 240 ppm.

21. Use according to any one of claims 15 to 19, characterized in that the composition comprises, for topical administration, a compound derived from titanium and from fluorine in an amount such that the titanium content is greater than 0.001% by weight, preferably between 0.01 and 0.1% by weight, with respect to the total weight of the said composition.

22. Use according to any one of claims 15 to 21, characterized in that it furthermore comprises an additional fluorinated compound, in particular a fluorine salt, for example sodium fluoride or sodium monofluorophosphate, in an amount varying from approximately 50 ppm to approximately 1500 ppm, preferably from approximately 100 ppm to approximately 500 ppm.

23. Use according to any one of claims 15 to 22, characterized in that the composition is administered by the topical route in the form of a dentifrice, of a powder to be diluted, of a spray, of a chewing gum, of a pastille to be sucked, of a gel, of a buccal implant, such as a patch, of a mouthwash or of a solution.

24. Process for protecting apatite-based materials from coloration, comprising the stage consisting in applying, to the apatite-based material, a composition comprising a titanium and fluorine derivative such as defined according to any one of claims 15 to 23, said composition possessing, in the solubilized state, a pH less than or equal to 6.

25. The process according to claim 24, further characterized in that, prior to the application of the composition, a treatment step with an acidic or demineralizing compound is carried out.

26. The process according to claim 25, further characterized in that the acidic or demineralizing compound is, in particular, citric acid, lactic acid, phosphoric acid, tartaric acid.
